(19) United States
(12) Patent Application Publication
(10) Pub. No.: US 2005/0182003 A1
(43) Pub. Date: Aug. 18, 2005

(54) Drugs to be used in gene therapy for recessively transmitted genetic disease

(76) Inventors: Masayuki Amagai, Shinjuku-ku, Tokyo (JP); Takeji Nishikawa, Tokyo (JP); Manabu Ohyama, Tokyo (JP)

Correspondence Address: MORGAN & FINNEGAN, L.L.P., 3 WORLD FINANCIAL CENTER, NEW YORK, NY, 10281-2101, US

(21) Appl. No.: 10/511,693

(22) PCT Filed: Apr. 15, 2003
(86) PCT No.: PCT/JP03/04765

(30) Foreign Application Priority Data
Apr. 15, 2002 (JP) 2002-112722

(51) Int. Cl. A61K 48/00; A61K 39/395

(52) U.S. Cl. 514/44; 424/93.2; 424/144.1

(57) ABSTRACT

The present invention provides a remedy to be used in gene therapy that can suppress immune response to a transgene product in gene therapy of genetic diseases such as recessive genetic diseases, or a method for treating genetic diseases such as recessive genetic diseases with the use of the remedy to be used in gene therapy. A remedy to be used in gene therapy is prepared with an immunosuppressive agent and a gene responsible for recessive genetic diseases such as recessive genetic epidermolysis bullosa hereditaria dystrophica, junctional epidermolysis bullosa hereditaria, hemidesmosome. epidermolysis bullosa hereditaria, ichthyosis congenita. As for the above-mentioned immunosuppressive agent, cyclosporin and anti-CD40L antibody can be exemplified. Moreover, the gene responsible for genetic diseases above mentioned can be used in a form of viral vector, and a form of naked DNA.

### TECHNICAL FIELD

The present invention relates to a remedy to be used in gene therapy that can suppress immune response to a transgene product in a gene therapy of genetic diseases such as autosomal recessive genetic diseases, or a method for treating genetic diseases such as autosomal recessive genetic diseases using the remedy to be used in gene therapy.

### BACKGROUND ART

Recently, genetic diagnosis has become reality in the field of dermatology wherein various genetic diseases exist, prenatal diagnosis by genetic diagnosis is actually made, and effects in basic medicine is now returning to clinical level. Presently, as a next step, a development of method for treating radically a genetic defect is anticipated. As for realizing gene therapy, there are various problems to gene expression itself such as development of effective gene transfer method, continuous gene expression method or the like. However, a clue to solve the problem is gradually coming up, due to development of various viral vectors.

However, as an object that had not been noticed heretofore and that must be overcome at the time of actual clinical application, there is a problem of immune response to the gene product extraneously introduced. In case of autosomal recessive genetic disease, as the gene responsible product(especially in case of extracellular constitutive protein) is deficient from the stage of ontogenesis of an affected patient, the patient's immune system has not met the protein at the stage when the immune system develops and differentiates. In other words, the immunological tolerance to the protein is not established in the patient's body. Therefore, when the gene therapy introducing extraneously a correct gene is carried out to correct symptoms due to genetic deficiency, it might happen that an immune response occur while the body immediately recognizes the gene product as a foreign substance, and that the therapeutic effect of gene therapy is reduced.

The necessity of suppressing the immune response to the transgene product are recently recognized gradually, however, up to now, it is only reported with the immune response to vectors, in gene therapy to organs besides skin, mainly with the use of viral vector (for example, Published Japanese Translation of PCT International Application No. 10-507758, Published Japanese Translation of PCT International Application No. 2001-512142, etc.), and sufficient investigation had not been made yet. Moreover, as for a method for suppressing the immune response, methods such as establishment of immunological tolerance to gene product, use of immunosuppressive agents, binding inhibition on surface molecules of immune response cells necessary for the establishment of immune response have been tried though none have been established up to now.

Prior arts to the present invention are cited in the following.
1) Amagai M, Hashimoto T, Shimizu N, and Nishikawa T: Absorption of pathogenic autoantibodies by the extracellular domain of pemphigus vulgarisantigen (Dsg3) produced by baculovirus. J Clin Invest 94:59-67, 1994
2) Amagai M, Klaus-Kovtun V, and Stanley J R: Autoantibodies against a novel epithelial cadherin in pemphigus vulgaris, a disease of cell adhesion. Cell 67:869-877, 1991
3) Amagai M, Koch P J, Nishikawa T, and Stanley J R: Pemphigus vulgaris antigen (Desmoglein 3) is localized in the lower epidermis, the site of blister formation in patients. J Invest Dermatol 106:351-355, 1996
4) Amagai M, Tsunoda K, Suzuki H, Nishifuji K, Koyasu S, and Nishikawa T: Use of autoantigen-knockout mice in developing an active autoimmune disease model for pemphigus. J Clin Invest 105:625-631, 2000
5) Chen M, O'Toole E A, Muellenhoff M, Medina E, Kasahara N, and Woodley D T: Development and characterization of a recombinant truncated type Vlll collagen “minigene”. Implication for gene therapy of dystrophic epidermolysis bullosa. J Biol Chem 275:24429-24435, 2000
6) Choate K A, Medalie D A, Morgan J R, and Khavari P A: Corrective gene transfer in the human skin disorder lammelar ichthyosis. Nat Med 2:1263-1267, 1996
7) Christensen R, Jensen U B, and Jensen T G: Cutaneous gene therapy-an update. Histochem Cell Biol 115:73-82, 2001
8) Dai Y, Schwarz E M, Gu D, Zhang W-W, Sarvetnick N, and Verma I M: Cellular and humoral immune responses to adenoviral vectors containing factor lX gene: Tolerization of factor lX and vector antigens allows for long-term expression. Proc Natl Acad Sci 92:1401-1405, 1995
9) Datta S K, and Kalled S L: CD40-CD40 ligand interaction in autoimmunedisease. Arthritis Rheum 40:1735-1745, 1997
10) Dellambra E, Vailly J, Pellegrini G, Bondanza S, Golisano O, Macchia C, Zambruno G, Meneguzzi G, and De Luca M: Corrective transduction of human epidermal stem cells in laminin-5-dependent junctional epidermolysis bullosa. Hum Gene Ther 9:1359-1370, 1998
11) Freiberg R A, Choate K A, Deng H, Alperin E S, Shapiro L J, and Khavari P A: A model of corrective gene transfer in X-linked ichthyosis. Hum Mol Genet 6:927-933, 1997
12) Greenhalgh D A, Rothnagel J A, and Roop D R: Epidermis: An attractive target tissue for gene therapy. J Invest Dermatol 103:63S-69S,1994
13) Hengge U, Chan E F, Foster R A, Walker P S, and Vogel J C: Cytokine gene expression in epidermis with biological effects following injection of naked DNA. Nat Genet 10:161-166, 1995
14) Hengge U R, Walker P S, and Vogel J C: Expression of naked DNA in human, pig, and mouse skin. J Clin Invest in press, 1996
15) Ilan Y, Prakash R, Davidson A, Jona V, Droguette G, Horwitz M S, Chowdhury N R, and Chowdhury J R: Oral tolerization to adenoviral antigens permits long-term gene expression using recombinant adenoviral vectors. JClin Invest 99:1098-1106, 1997
16) Jensen T G, Jensen U B, Jensen P K, Ibsen H H, Brandrup F, Ballabio A, and Bolund L: Correction of steroid sulphatese deficiency by gene transfer into basal cells of tissue-cultured epidermis from patients with recessive X-linked ichthyosis. Exp Cell Res 209:392-397, 1993
17) Katsumi A, Emi N, Abe A, Hasegawa Y, Ito M, and Saito H: Humonal and cellular immunity to an encoded protein induced by direct DNA injection. Hum Gene Ther 5:, 1994
18) Kay M A, Hotlterman A X, Meuse L, Allen G, Ochs H D, Linsley P S, and Wilson C B: Long-term hepatic adenovirus-mediated gene expression in mice following CTLA4Ig administration. Nat Genet 11:191-197, 1995
19) Khavari P A: Gene therapy for genetic skin disease. J Invest Dermatol 110:462-467, 1998
20) Khavari P A: Genetic correction of inherited epidermal disorders. Hum Gene Ther 11:2277-2282, 2000
21) Koch P J, Mahoney M G, Ishikawa H, Pulkkinen L, Uitto J, Shultz L, Murphy G F, Whitaker-Menezes D, and Stanley J R: Targeted disruption of the pemphigus vulgaris antigen (desmoglein 3) gene in mice causes loss of keratinocyte cell adhesion with a phenotype similar to pemphigus vulgaris. J Cell Biol 137:1091-1102, 1997
22) Larregina A T, and Falo L D: Generating and regulating immune responses through cutaneous gene delivery. Hum Gene Ther 11:2301-2305, 2000
23) Morral N, O'neal W, Zhou H, Langston C, and Beaudet A: Immune response to reporter proteins and high viral dose limit duration of expression with adenoviral vectors: comparison of E2a wild type and E2a deleted vectors. Hum Gene Ther 8:1275-1286, 1997
24) Ohyama M, Amagai M, Tsunoda K, Ota T, Koyasu S, Hata J, Umezawa A, and Nishikawa T: Immunologic and histopathologic characterization of active disease mouse model for pemphigus vulgaris. J Invest Dermatol 118:199-204, 2002
25) Seitz C S, Giudice G I, Balding S D, Marinkovich M P, and Khavari P A:BP180 gene delivery in junctional epidermolysis bullosa. Gene Ther 6:42-47, 1999 Spirito F, Meneguzzi G, Danos O, and Mezzina M: Cutaneous gene transfer and therapy; the present and the future. J Gene Med 3:21-31, 2001
26) Stein C S, Martins I, and Davidson B L: Long-term reversal of hypercholesterolemia in low density lipoprotein receptor (LDLR)-deficient mice by adenovirus-mediated LDLR gene transfer combined with CD154 blokade. J Gene Med 2:41-51, 2000
27) Tripathy S K, Black H B, Goldwasser E, and Leiden J M: Immune responses to transgene-encoded proteins limit the stability of gene expresson after injection of replication-defective adenovirus vectors. Nat Med 2:54 5-550, 1996
28) Uitto J, and Pulkkinen L: The genodermatoses: candidate disease for gene therapy. Hum Gene Ther 11:2267-2275, 2000
29) Vaily J, Gagnoux-Palacios L, Del'Ambra E, Romero C, Pinola M, Zambruno G, De Luca M, Ortonne J-P, and Meneguzzi G: Corrective gene transfer of keratinocytes from patients with junctional epidermolysis bullosa restores assembly of hemidesmosomes in reconstructed epithelia. Gene Ther 1322-1322, 1998
30) Vogel J C: Keratinocyte gene therapy. Arch Dermatol 129:1478-1483, 1993
31) Vogel J C: Nonviral skin gene therapy. Hum Gene Ther 11:2253-2259, 2000
32) Yamada A, and Sayegh M H: The CD154-CD40 costimulatory pathway in transplantation. Transplantation 73:S36-S39, 2002
33) Yang Y, Su Q, Grewal I S, Schilz R, Flavell R A, and Wilson J M: Transient subversion of CD40 ligand function diminishes immune response to adenovirus vectors in mouse liver and lung tissues. 1996 70:6370-6377, 1996
34) Yao S N, Farjo A, Roessler B J, Davidson B L, and Kurachi K: Adenovirus-mediated transfer of huma factor lX gene in iummuno deficient and normal mice. Viral Immunol 9:141-153, 1996
35) Zeng L, Sarasin A, and Mezzina M: Retrovirus-mediated DNA repair gene transfer into xeroderma pigmentosum cells. Cell Biol Toxicol 14:105-110, 1998

For treatment of congenital genetic diseases wherein the deficient gene is known, it is necessary to perform gene therapy and supplement the deficient gene. However, as immunological tolerance to gene product of the deficient gene is not established in patient with the deficient gene, when the correct gene is introduced, it would initiate immune response to the gene product, induce autoimmunity, and the gene introduced would not function accurately. The object of the present invention is to provide a remedy to be used in gene therapy that can suppress immune response to transgene product in gene therapy of genetic diseases such as autosomal recessive genetic diseases, or a method for treating genetic diseases such as autosomal recessive genetic diseases by using the remedy to be used in gene therapy.

As for the estimation of immune response to a transgene product in a conventional gene therapy and for making attempts to suppress the immune response, there were problems as follows: 1) though the possibility of an inappropriate immune response to occur increases significantly when a deficient gene is introduced in a body wherein a transgene product is completely deficient at the stage of development, that is a body having a phenotype of autosomal recessive inheritance, such body having deficient gene is not targeted; and gene is introduced only in test subject-body, and the method for suppressing the immune response to the product is estimated. 2) as for the gene transfer method, a method using a viral vector, mainly an adenoviral vector is carried out, and estimation of immune response to only transgene product, not associated with the immune response of the body to be treated to viral vector, or suppression of response is not made. 3) in a strain using gene transfer method without using virus, gene expression as stable as to estimate immune response to transgene product, which can be the subject for applying suppressing method, cannot be obtained. 4) estimation of method of suppressing immune response, being effective and having small side effect, is not sufficient.

With the view of these problems of prior arts, the present inventors carried out gene therapy to introduce a correct gene to a knockout mouse whose skin component is deficient, confirmed whether immune response to the gene product was present or not, generated a strain of the experimental animal model that can be used to analyze the immune response in gene therapy, and investigated various methods such as establishing immunological tolerance to suppress immune response to exogenous gene product, with the following concept: 1) To select an animal wherein a single gene whose function is sufficiently analyzed is deficient and whose pathophysiology is sufficiently analyzed, when selecting animal disease model to be the target of gene therapy. 2) To estimate the immune response to only transgene product and a suppressing method thereof by using a gene transfer method without using virus. 3) To establish an appropriate strain simulating a condition that a gene has been successfully introduced, when a stable gene expression can not be obtained. 4) To realize an effective suppression of immune response by using biologically active substances that block biologically the in vivo pathway indispensable for the initiation of immune response, without depending on remedies and the like.

Specifically, a Dsg3-knockout mouse (Dsg3^{-/-}) wherein Desmoglein 3 (Dsg3), a component of epidermis and a cell membrane protein, is deficient was used. As the mouse lacks Dsg3 which is an cell-to-cell adhesion molecule, oral blister/erosion is formed and telogen hair loss occur. As a gene therapy to the mouse, a mouse-Dsg3 gene, having correct gene arrangement integrated-to an expression vector, was introduced in epidermal cells by Naked DNA injection method, and it was confirmed that the introduced Dsg3 gene can be expressed in epidermis and hair follicle. Moreover, it was confirmed that antibody production to the expressed-Dsg3 was maintained, but by introducing anti-CD40L monoclonal antibody as an immunosuppressive agent before gene therapy, immune response to the transgene product Dsg3 having possibility of reducing therapeutic effect of gene therapy is effectively suppressed. The present invention has been completed based on these knowledge.

### DISCLOSURE OF THE INVENTION

In other words, the present invention relates to: a remedy to be used in gene therapy of genetic diseases comprising an immunosuppressive agent and a gene responsible for genetic diseases (“1”); the remedy to be used in gene therapy of genetic diseases according to “1”, wherein the remedy contains a immunosuppressive agent and a gene responsible for genetic diseases (“2”); the remedy to be used in gene therapy of genetic diseases according to “1” or “2”, wherein the immunosuppressive agent has as an active ingredient an antagonist inhibiting the interaction between the receptor CD40L mediating the contact-dependent helper effector function on the surface of T cells and the receptor CD40 on the surface of antigen presenting cells (“3”); the remedy to be used in gene therapy of genetic diseases according to “3”, wherein the antagonist inhibiting the interaction is an anti-CD40L antibody (“4”); the remedy to be used in gene therapy of genetic diseases according to any of “1” to “4”, wherein the gene responsible for genetic diseases has a form of a viral vector or a naked DNA (“5”); the remedy to be used in gene therapy of genetic diseases according to any of “1” to “5”, wherein the genetic disease is a recessive genetic disease (“6”); and the remedy to be used in gene therapy of genetic diseases according to “6”, wherein the recessive genetic disease is an autosomal recessive genetic disease (“7”).

Moreover, the present invention relates to a method for treating genetic diseases wherein the remedy to be used in gene therapy of genetic diseases according to any of “1” to “7” is used (“8”); and the method for treating genetic diseases according to “8”, wherein the genetic diseases are pemphigus, recessive genetic epidermolysis bullosa heredetaria dystrophica, junctional epidermolysis heredetaria bullosa, hemidesmosome epidermolysis bullosa hereditaria or ichthyosis congenita (“9”).

### BRIEF EXPLANATION OF THE DRAWINGS

FIG. 1 is a set of pictures showing the results of immunofluorescence staining method, showing the expression of Dsg3 after the introduction of pcDNA:mDsg3 to the Dsg3^{-/-} mouse epidermis by Naked DNA injection method.

a: pcDNA:mDsg3-introduced site (arrow)

b: pcDNA-introduced site (scale:50 μm)

FIG. 2 is a graph showing the result of ELISA method showing the production of IgG antibody against the transgene product Dsg3 (vertical axe: OD level of ELISA method; horizontal axe: number of days from the initiation of the treatment).

FIG. 3 is a picture showing the results of immunofluorescence staining method, showing the binding of anti-Dsg3 IgG antibody generated by gene therapy to Dsg3 expressed by gene therapy (arrow) (scale:50 μm).

FIG. 4 is a picture showing Dsg3^{-/-} mouse wherein Dsg3^{+/+} mouse skin graft survives (arrow).

FIG. 5 is a graph showing the results of ELISA method, showing the anti-Dsg3 IgG antibody production in Dsg3^{+/+} graft strain. For the hamster-IgG administered group as a control, anti-Dsg3 IgG antibody production was observed in approximately 2 weeks (full line), while the IgG production was significantly suppressed for the MR1-administered group(dotted line).

(vertical axe: OD level of mouse Dsg3 IgG ELISA; horizontal axe: number of days after skin graft).

FIG. 6 is a picture showing the results of immunofluorescence staining method, showing the in vivo binding of anti-Dsg3 IgG antibody generated in Dsg3^{+/+} graft strain to Dsg3 molecule in vivo.

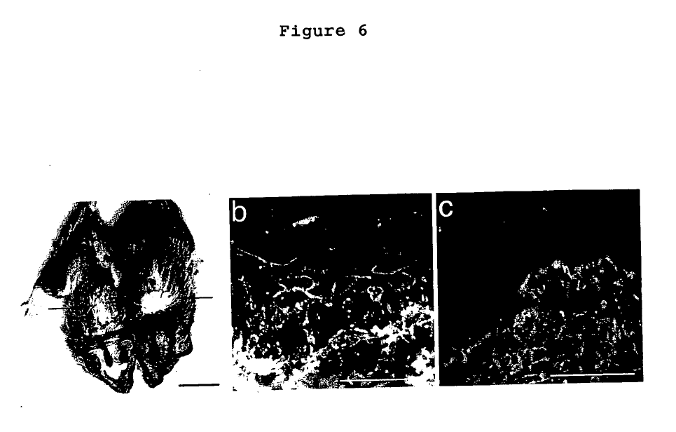

a: survival of graft skin in MR1-administered Dsg3^{-/-} mouse group (FIG. 6a left); and survival of re-grafted skin 5 to 7 days after skin re-graft (FIG. 6a right) (scale:1 cm).

b: In hamster-IgG administered group, deposit of IgG between epidermis cells in graft skin is observed (scale: 50 μg).

c: In MR1-administered group, deposit of IgG between epidermis cells in graft skin is not observed (scale: 50 μm).

### BEST MODE OF CARRYING OUT THE INVENTION

As for a remedy of the present invention, there is no specific limitation as long as it is a remedy to be used for gene therapy of genetic diseases comprising an immunosuppressive agent of the present invention and a gene responsible for genetic diseases. The genetic diseases mentioned herein relate to genetic diseases wherein immunological tolerance to gene product of the deficient gene is not established when gene therapy to supplement the deficient gene is applied, and recessive genetic diseases such as autosomal recessive genetic diseases or sex-linked genetic diseases can be exemplified. Examples of the autosomal recessive genetic diseases include: pemphigus, recessive genetic epidermolysis bullosa hereditaria dystrophica, junctional epidermolysis bullosa hereditaria, hemidesmosome epidermolysis bullosa hereditaria, ichthyosis congenita, albinism, Tay-Sachs disease, Wilson disease, Cystic Fibrosis, Phenylketonuria, type I glycogenosis, galactosemia. Examples of sex-linked genetic diseases include achromatopsia, hemophilia A, Duchenne type muscular dystrophy and the like.

As for the immunosuppressive agent mentioned above, there is no specific limitation as long as it can suppress the immune response initiated by the gene product which was deficient when gene therapy was applied, including immunosuppresive agents publicly known. It can be preferably exemplified by an antagonist inhibiting interaction between receptor CD40L mediating contact-dependent helper effector function on the surface of T cells and receptor CD40 on the surface of antigen presenting cells, besides Cyclosporin A, tacrolimus (FK506), Cyclophosphamide, azathioprine, mizoribine, steroid, Methotrexate, antihistamine and the like. As for such antagonists, examples include: antibody against CD40L (e.g. monoclonal antibody against CD40L), fragment of antibody against CD40L (e.g. fragment of Fab or (Fab′)₂), chimeric antibody, humanized antibody, and soluble CD40 or soluble CD40L and fragment thereof, or other compounds inhibiting the interaction between CD40L and CD 40.

The gene responsible for genetic diseases mentioned above are generally used in form of viral vectors, in form of naked DNA, liposome subsumption form and the like, and the gene responsible can be genomic DNA, cDNA, mDNA or synthesized DNA. The viral vectors mentioned above can be prepared based on DNA or RNA virus, while the origin of viral species is not specifically limited, and it can be any type of viral vector such as MOMLV vector, helpes viral vector, adenoviral vector, adeno-associated viral vector, HIV vector, Sendai viral vector, vaccinia viral vector.

For example, as for the adenoviral vector, it is preferable that ITR (inverted terminal repeat) and envelope repeat are included, and a whole of or a part of E1 adenovirus region is deficient. Moreover, a whole of or a part of E3 adenovirus region can be deficient, however it is preferable that a part of E3 region that encodes glicoprotein gp19k is maintained. Furthermore, as HIV viral vector incorporates the introduced nucleic acid into the chromosome, it can express continuously a drug-gene which is the nucleic acid. Moreover, as HIV vector is able to transfer genes selectively to CD4-positive T cells, which are molecules on the surface of cells, and also to integrate to chromosomes even during resting stage when cells are not dividing. Thus, for example, by using peusdotype HIV viral vector wherein Env protein being a HIV coat protein was substituted for VSV-G protein, which is a coat protein of Vesicular stomatitis virus, it would be possible to introduce drug-genes effectively to any type of cells in resting stage, such as bone marrow stem cells, hematopoietic stem cells, neuron and muscle cells.

As a naked DNA form, a form of plasmid DNA can be preferably exemplified, and as for such plasmid, expression vector plasmid for animal cells which are publicly known can be exemplified. As for the vector plasmid, those including viral promoter, for example CMV (cytomegalovirus) promoter, RSV (Rous Sarcoma Virus) promoter, HSV-1 virus TK-gene promoter, SV40 (Simian Virus 40) early promoter, adenoviral MLP (major late promoter) promoter, are preferable. Furthermore, it can include marker gene that can select or identify transfected cells. As for such marker genes, examples include: neo gene giving resistance to antibiotic G418 (encoding neomycin phosphotransferase), dhfr (dihydrofolate reductase) gene, CAT (Chloramphenicol AcetylTransferase) gene, pac (puromycin acetyltransferase) gene, gpt(xanthine guanine phophoribosyl transferase) gene.

As it is mentioned above, the remedy to be used in gene therapy for genetic diseases of the present invention comprises an immunosuppressive agent and a gene responsible for genetic diseases. In case the immunosuppressive agent as for example CD40L comprises protein or peptide, it is possible to make a remedy to be used in gene therapy of genetic diseases comprising an immunosuppressive agent and a gene responsible for genetic diseases, by co-integrating DNA encoding the protein or peptide and a gene responsible to viral vector or plasmid vector. The remedy to be used for gene therapy of the present invention can be administered to patients having genetic diseases, as well as to patients who are predicted to develop genetic diseases.

The method for treating genetic diseases of the present invention is carried out by using once or more the remedy to be used for gene therapy of genetic diseases of-the present invention mentioned above, and it is possible to administer the immunosuppressive agent and the gene responsible for genetic diseases simultaneously, or to administer the immunosuppressive agent after carrying out gene therapy with the use of the gene responsible for genetic diseases, and the site to administer can be different. The administration can be done either parenterally by injection and the like or orally, subcutaneously, intravenously, intramuscularly, peritonealy, intrasynovially, intrapulmonary, intragastrically, intranasally, intratracheally and so on. The dosage form of the remedy to be use in gene therapy of the present invention is selected appropriately according to the administration method, and for example, as for pharmaceutical composition adequate for injection, sterilized solution (if soluble) or sterilized powder to prepare immediately dispersion solution and sterilized injection solution or a dispersion solution, or the like. The dosage is the amount sufficient to expect therapeutic effect, and can be selected appropriately according to patient's age, sex, sensibility to drugs, administrating method, history of diseases and so on.

The present invention will be explained in detail in the following, however the technical scope of the present invention will not be limited to these examples.

### EXAMPLE 1

### Introduction of Dsg3 Gene to Dsg3^{-/-} Mouse Epidermis by Naked DNA Injection Method

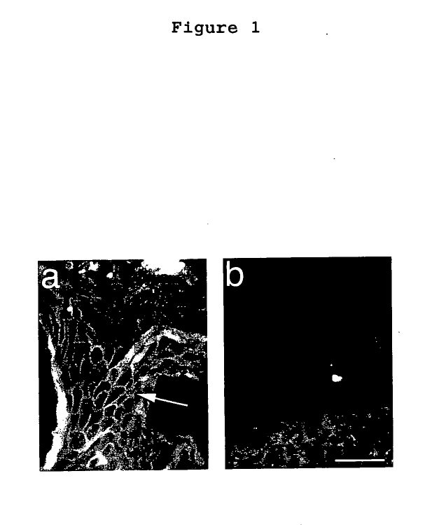

Dsg3 is a protein on the surface of cells, being a component of desmosome which is an adhering junction between epithelial cells. Mouse Dsg3 (mDsg3) was subcloned to pcDNA, an expression vector using CMV promoter, to prepare a plasmid (pcDNA:mDsg3). The plasmid was diluted with PBS to a concentration of 1-10 μg/μl, and the solution was injected into Dsg3^{-/-} mouse superficial dermis, by Naked DNA injection method, reported previously. 18 hours after the injection, the skin of the plasmid-introduced site was biopsied, observed by immunofluorescence staining method with the use of anti-mDsg3 monoclonal antibody and MDsg3 expression was observed between epidermal cells of pcDNA:mDsg3-introduced site (FIG. 1a). Such observation was not confirmed in pcDNA-introduced site as control (FIG. 1b). From the above, it has been clarified that Dsg3 could be introduced in ordinary expression site in mouse individual by naked DNA injection method.

### EXAMPLE 2

### Investigation of anti-Dsg3 IgG Antibody Production by Transferring Dsg3 Gene

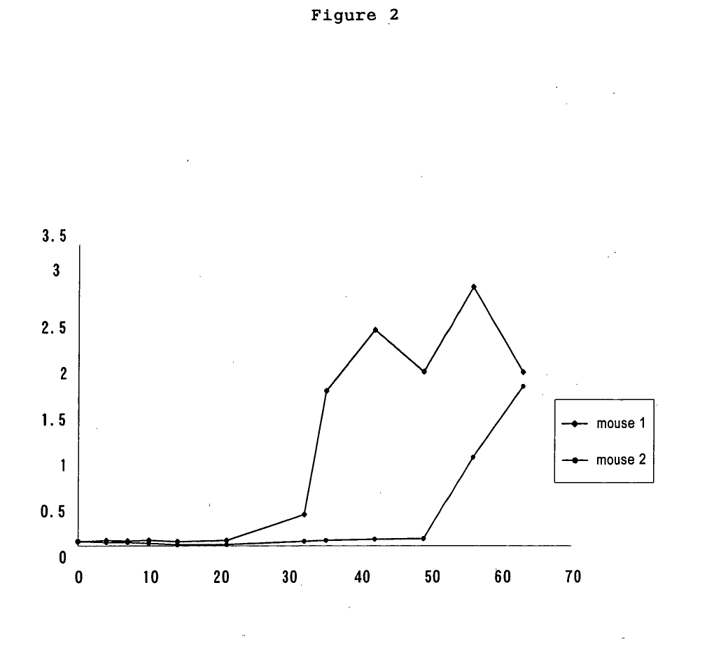

It was investigated whether antibodies against transgene product were produced or not in Dsg3^{-/-} mouse wherein Dsg3 gene is introduced to the epidermis by naked DNA injection method, by enzyme-linked immunosorbent assay (ELISA) method with the use of recombinant mDsg3 produced by the baculovirus expression system. As a protocol of gene therapy, the following administrating methods was determined, and two Dsg 3^{-/-} mice per each protocol were used:
1) administrating pcDNA:mDsg3 once a week, by 50 μg/mouse;
2) administrating pcDNA:mDsg3 twice a week, by 50 μg/mouse;
3) administrating pcDNA:mDsg3 once a week, by 100 μg/mouse;
4) administrating pcDNA:mDsg3 twice a week, by 100 μg/mouse;
5) administrating pcDNA:mDsg3 only once, by 200 μg/mouse;
6) administrating pcDNA:mDsg3 once every two weeks, by 200 μg/mouse. Among these, generation of anti-Dsg3 IgG antibody was observed in serum of one of the Dsg3^{-/-} mice wherein gene was introduced according to any administration methods of 2) to 4), and in serum of two mice administered a gene according to 6). Especially, antibody generation was confirmed to continue for a long period of 60 days, for the two mice wherein gene was introduced with the method 6) (FIG. 2).

### EXAMPLE 3

### Investigation of Binding of anti-Dsg3 IgG Antibody Generated by Gene Therapy to Transgene Product

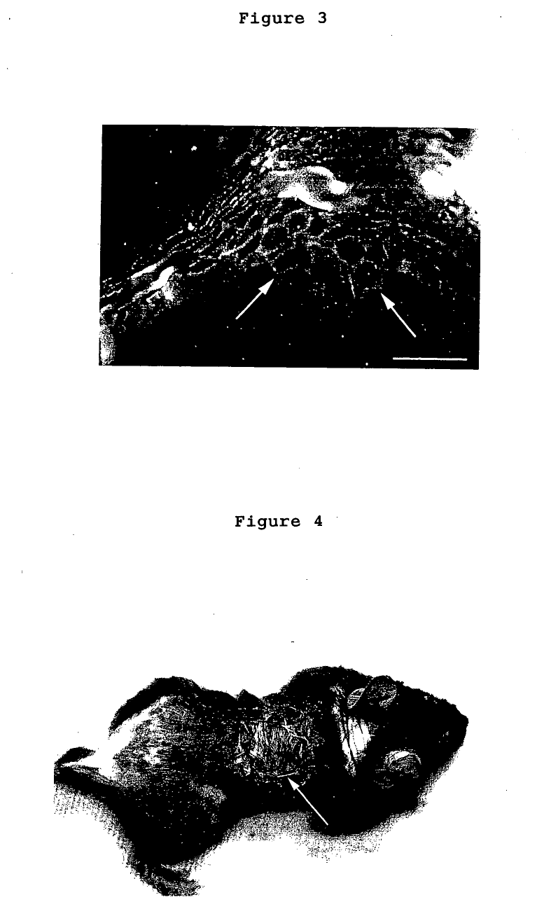

It was investigated whether anti-Dsg3 antibody generated by Dsg3 gene transfer actually recognizes Dsg3 expressed by gene transfer in vivo or not. After transferring gene to mouse epidermis wherein increased antibody titer was observed by ELISA by the aforementioned method, the site of treatment was subjected to biopsy. The obtained tissue section was observed by immunofluorescence staining method with the use of anti-mouse IgG polyclonal antibody, and deposit of IgG between epidermis cells was observed accordingly to the gene transferred site (FIG. 3). From the above, a possibility that the antibody to the transgene product generated as side product of gene therapy would actually bind to transgene product was suggested.

### EXAMPLE 4

### Experiment Strain Simulating Stable gene Transfer; Establishment of a Strain to Transplant Dsg3^{+/+} Mouse Skin to a Dsg3^{-/-} Mouse

From the investigation above mentioned, it was shown that by introducing Dsg3 gene to Dsg3^{-/-} mouse, antibody against transgene product generates, and that the generated antibody can recognize transgene product. However, by using the naked DNA injection method used for this investigation, a relatively stable gene transfer can be expected for animals having thick epidermis such as humans and pigs, while no stable gene transfer can be expected for mouse, as its epidermis is very thin. Therefore, for further investigation of immune response in gene therapy, a strain to transplant Dsg3^{+/+} mouse skin to Dsg3^{-/-} mouse was established. In mouse wherein skin graft survived (FIG. 4), it is believed that a condition wherein Dsg3 was transferred successfully to epidermis was imitated locally. Moreover, it was confirmed with the investigation by ELISA method, described above, that in this strain (hereinafter referred to as Dsg3^{+/+} graft strain), anti-Dsg3 IgG antibody was generated two weeks after skin graft in the same manner as when gene was transferred by naked DNA injection method. Therefore, in the following investigation, immune response in gene therapy and the suppressing method thereof were estimated by using the stable Dsg3^{+/+} graft strain.

### EXAMPLE 5

### Investigation of Suppression of anti-Dsg3 IgG Antibody Production in Dsg3^{+/+} Graft Strain with the use of Anti-CD40L Monoclonal Antibody

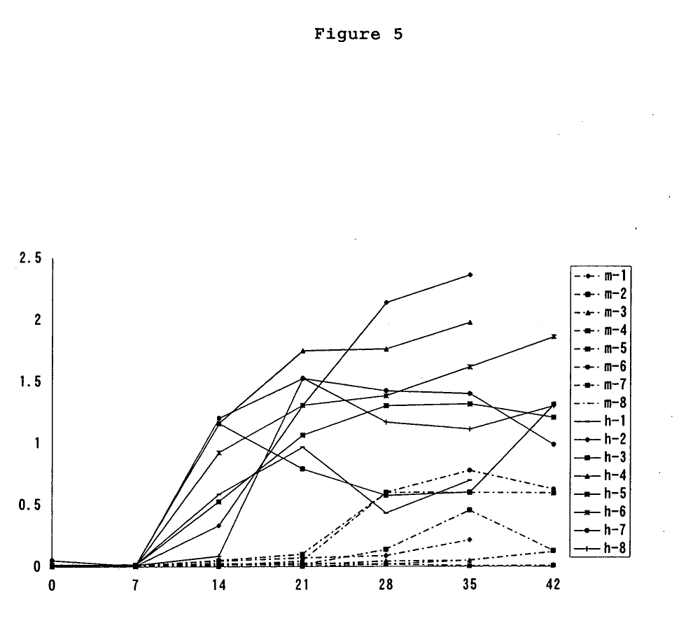

As for a method for suppressing antigen-specific immune response, various methods have been already reported. At first, the present inventors had planned to suppress immune response by oral tolerance, and prepared Dsg3 protein with the use of E. Coli expression vector, administered orally to a mouse, but failed to obtain successful results. Therefore, they tried to suppress the anti-Dsg3 antibody generation in Dsg3^{+/+} graft strain by inhibiting the binding of CD40 and CD40L, which are playing an important role to the establishment of immune response. CD40L is a type II cell membrane-penetrating protein, expressed transiently in activated T cells having received antigen stimulation and CD40, its receptor, is expressed in B cells, dendritic cells, monocyte/macrophage, endothelial cells and the like. The CD40-CD40L binding not only plays an important role in cellular immunity by promoting cytokine production and the like, but it is clarified to be quite important for proliferation of B cells, antibody production and the like. Thus, attempts have been made to suppress immune response in autoimmune diseases, organ transplantation and the like by using anti-CD40L monoclonal antibody inhibiting this binding. As mentioned above, in Dsg3^{+/+} graft strain, anti-Dsg3 IgG antibody generation, which is detected in serum by ELISA method, is observed approximately two weeks after skin graft, in all mice that underwent skin graft. However, when MRI, an hamster-derived anti-CD40L antibody, is administered intraperitoneally with the following schedule, it was suggested by ELISA method that the antibody generation is significantly suppressed (FIG. 5): at day 0 (1000 μg/mouse), day 2, day 4, day 7, day 14, day 21, day 28 (500 μg/mouse) after skin graft.

### EXAMPLE 6

### Estimation of Binding of Anti-Dsg3 IgG Antibody Generated in Dsg3^{+/+} Graft Strain to Dsg3 Molecule in vivo)

To demonstrate that the anti-Dsg3 antibody generated in Dsg3^{+/+} graft strain actually binds to Dsg3 molecule in vivo, the graft skin section was subjected to biopsy 4-5 weeks after skin graft, and deposit of IgG to intracellular epidermis was estimated by immunofluorescence staining method with the use of anti-mouse IgG polyclonal antibody. In MR1-administered Dsg3^{-/-} mouse group, the graft skin section continues to survive (FIG. 6a, left), while in hamster IgG-administered group as control, the graft skin section was rejected in approximately 3 weeks. Therefore, for this group, when the skin graft section dropped off, skin graft was conducted again, and biopsy was taken 5-7 days after the skin re-graft, when the survival of re-grafted skin section was confirmed (FIG. 6a, right). As for control group, obvious deposit of IgG between intracellular epidermis was confirmed (FIG. 6b), while as for MR1-administered group, such deposit was not obvious (FIG. 6c). From the above, it was suggested that MR1 suppresses anti-Dsg3 IgG antibody generation in Dsg3^{+/+} graft strain, both in vivo and in vitro.

### INDUSTRIAL APPLICABILITY

According to the present invention, it has been clarified that by introducing anti-CD40L monoclonal antibody before the therapy, immune response to transgene product that has possibility to decrease treatment effect of gene therapy which has been successful in mouse model having recessive genetic diseases was suppressed effectively. In other words, according to the present invention, in order to accomplish gene therapy to recessive genetic diseases, the suppression of the immune response against the immune product of transgene product is necessary, and it was shown that anti-CD40L monoclonal antibody was useful to the suppression.

1. A remedy to be used in gene therapy of genetic diseases comprising an immunosuppressive agent and a gene responsible for genetic diseases.

2. The remedy to be used in gene therapy of genetic diseases according to claim 1, wherein the remedy a consists of an immunosuppressive agent and a gene responsible for genetic diseases.

3. The remedy to be used in gene therapy of genetic diseases according to claim 1 or 2, wherein the immunosuppressive agent has as an active ingredient an antagonist which inhibits the interaction between a CD40 receptor ligand, CD40L, and a CD40 receptor on the surface of antigen presenting cells, wherein the CD40L mediates a contact-dependent helper effector function on the surface of T cells.

4. The remedy to be used in gene therapy of genetic diseases according to claim 3, wherein the antagonist inhibiting the interaction is an anti-CD40L antibody.

5. The remedy to be used in gene therapy of genetic diseases according to claim 1 or 2, wherein the gene responsible for genetic diseases has a form of a viral vector or a naked DNA.

6. The remedy to be used in gene therapy of genetic diseases according to claim 1 or 2, wherein the genetic disease is a recessive genetic disease.

7. The remedy to be used in gene therapy of genetic diseases according to claim 6, wherein the recessive genetic disease is an autosomal recessive genetic disease.

8. A method for treating genetic diseases comprising administering a remedy comprising an immunosuppressive agent and a gene responsible for genetic diseases.

9. The method for treating genetic diseases according to claim 8, wherein the genetic diseases are pemphigus, recessive genetic epidermolysis bullosa heredetaria dystrophica, junctional epidermolysis heredetaria bullosa, hemidesmosome epidermolysis bullosa hereditaria or ichthyosis congenita.

10. The remedy to be used in gene therapy of genetic diseases according to claim 3, wherein the gene responsible for genetic diseases is a viral vector or a naked DNA.

11. The remedy to be used in gene therapy of genetic diseases according to claim 4, wherein the gene responsible for genetic diseases is a viral vector or a naked DNA.

12. The remedy to be used in gene therapy of genetic diseases according to claim 3, wherein the genetic disease is a recessive genetic disease.

13. The remedy to be used in gene therapy of genetic diseases according to claim 4, wherein the genetic disease is a recessive genetic disease.

14. The remedy to be used in gene therapy of genetic diseases according to claim 5, wherein the genetic disease is a recessive genetic disease.

15. The remedy to be used in gene therapy of genetic diseases according to claim 12, wherein the recessive genetic disease is an autosomal recessive genetic disease.

16. The remedy to be used in gene therapy of genetic diseases according to claim 13, wherein the recessive genetic disease is an autosomal recessive genetic disease.

17. The remedy to be used in gene therapy of genetic diseases according to claim 14, wherein the recessive genetic disease is an autosomal recessive genetic disease.

18. The method for treating genetic diseases according to claim 14, wherein the genetic diseases are pemphigus, recessive genetic epidermolysis bullosa heredetaria dystrophica, junctional epidermolysis heredetaria bullosa, hemidesmosome epidermolysis bullosa hereditaria or ichthyosis congenita.

19. The method for treating genetic diseases according to claim 8, comprising administering a remedy consisting of an immunosuppressive agent and a gene responsible for genetic diseases.

20. The method for treating genetic diseases according to claim 8 or 19, wherein the immunosuppressive agent has as an active ingredient an antagonist which inhibits the interaction between a CD40 receptor ligand, CD40L, wherein the CD40L mediates a contact-dependent helper effector function on the surface of T cells, and a CD40 receptor on the surface of antigen presenting cells.

21. The method for treating genetic diseases according to claim 20, wherein the antagonist inhibiting the interaction is an anti-CD40L antibody.

22. The method for treating genetic according to claim 8, wherein the gene responsible for genetic diseases has a form of a viral vector or a naked DNA.

23. The method for treating genetic according to claim 14, wherein the gene responsible for genetic diseases has a form of a viral vector or a naked DNA.

24. The method for treating genetic according to claim 20, wherein the gene responsible for genetic diseases has a form of a viral vector or a naked DNA.

25. The method for treating genetic according to claim 21, wherein the gene responsible for genetic diseases has a form of a viral vector or a naked DNA.

26. The method for treating genetic diseases according to claim 8, wherein the genetic disease is a recessive genetic disease.

27. The method for treating genetic diseases according to claim 14, wherein the genetic disease is a recessive genetic disease.

28. The method for treating genetic diseases according to claim 20, wherein the genetic disease is a recessive genetic disease.

29. The method for treating genetic diseases according to claim 21, wherein the genetic disease is a recessive genetic disease.

30. The method for treating genetic diseases according to claim 22, wherein the genetic disease is a recessive genetic disease.

31. The method for treating genetic diseases according-to claim 23, wherein the genetic disease is a recessive genetic disease.

32. The method for treating genetic diseases according to claim 24, wherein the genetic disease is a recessive genetic disease.

33. The method for treating genetic diseases according to claim 25, wherein the genetic disease is a recessive genetic disease.

34. The method for treating genetic diseases according to claim 26, wherein the recessive genetic disease is an autosomal recessive genetic disease.

35. The method for treating genetic diseases according to claim 27, wherein the recessive genetic disease is an autosomal recessive genetic disease.

36. The method for treating genetic diseases according to claim 28, wherein the recessive genetic disease is an autosomal recessive genetic disease.

37. The method for treating genetic diseases according to claim 29, wherein the recessive genetic disease is an autosomal recessive genetic disease.

38. The method for treating genetic diseases according to claim 30, wherein the recessive genetic disease is an autosomal recessive genetic disease.

39. The method for treating genetic diseases according to claim 31, wherein the recessive genetic disease is an autosomal recessive genetic disease.

40. The method for treating genetic diseases according to claim 32, wherein the recessive genetic disease is an autosomal recessive genetic disease.

41. The method for treating genetic diseases according to claim 33, wherein the recessive genetic disease is an autosomal recessive genetic disease.
